(19) United States
(12) Patent Application Publication
(10) Pub. No.: US 2025/0185503 A1
(43) Pub. Date: Jun. 5, 2025

(54) Nonlinear Optical Chromophores Containing Spirofluorene-Isophorone Bridging Groups, and Methods of Making and Using the Same

(71) Applicant: Lightwave Logic, Inc., Englewood, CO (US)

(72) Inventors: Ginelle A. Ramann, Centennial, CO (US); Barry L. Johnson, Castle Rock, CO (US)

(73) Assignee: Lightwave Logic, Inc., Englewood, CO (US)

(21) Appl. No.: 18/967,133

(22) Filed: Dec. 3, 2024

Related U.S. Application Data
(60) Provisional application No. 63/605,792, filed on Dec. 4, 2023.

(51) Int. Cl. H10K 85/60; C07D 405/08; C09B 23/01; C09K 9/02; G02F 1/355; G02F 1/361

(52) U.S. Cl. CPC H10K 85/652; C07D 405/08; C09B 23/0066; C09K 9/02; G02F 1/3558; G02F 1/3611; H10K 85/622; H10K 85/626; H10K 85/653; H10K 85/6572; C09K 2211/1007; C09K 2211/1011; C09K 2211/1014; C09K 2211/1018

(57) ABSTRACT

The present disclosure is directed, in general, to (1) nonlinear optical (NLO) chromophores containing spirofluorene-isophorone bridging groups, including (2) compositions/materials/resistive layers comprising NLO chromophores containing spirofluorene-isophorone bridging groups, and the methods of making the compositions/materials/resistive layers comprising NLO chromophores containing spirofluorene-isophorone bridging groups (e.g., methods of poling and/or drying, and the like), (3) uses of NLO chromophores containing spirofluorene-isophorone bridging groups in electro-optic devices (e.g., EOMs).

### CROSS-REFERENCE TO RELATED APPLICATIONS

This application claims the benefit of U.S. Provisional Application No. 63/605,792 filed on Dec. 4, 2023, the entire contents of which is incorporated herein by reference.

### BACKGROUND

The rise of silicon photonics has led to renewed interest in the use of electro-optic (EO) materials in next generation device applications. Materials with a strong EO response and high-speed phase modulation in thin film form are essential for low power and small footprint devices, including devices used in data acquisition systems, analog I/O modules, field transmitters, lab and field instrumentation, servo drive control modules, direct current (DC) power supply, alternating current (AC), and/or electronic load.

Generally, EO response reflects the change in a material's optical properties (e.g., refractive index) in response to an electrical field, and the strength of an EO response is correlated with the strength of the material's Pockels effect. The Pockels effect (or linear EO effect) is a directionally dependent linear variation in the refractive index of an optical medium that occurs in response to the application of an electric field. Macroscopically, the Pockels coefficient r relates the change in the index of refraction to an applied electric field as

$$n ( E ) = {n}_{0} - \frac{1}{2} {rn}_{0}^{3} E$$

where n₀ is the index of refraction under no field and n is the index of refraction under a given electrical field with the voltage equals to E. The applied electrical field shifts the electron cloud to the excited-state molecular orbitals, which alters the refractive index of the EO materials. In optical media, the Pockels effect causes changes in birefringence that vary in proportion to the strength of the applied electric field. The EO coefficient r₃₃ in units of pmV⁻¹ is the principal element of the linear Pockel's EO effect tensor and denotes the magnitude of refractive index shift (Δη) obtained for an applied low-frequency electric field.

EO materials generally fall into three categories: (1) liquid crystals, including ferroelectric liquid crystals, and/or organic liquid crystals having a linear structure with a central core that contains several collinear rings, a linear unsaturated linkage and two terminal chains, and the like; (2) inorganic crystals characterized by a lack of inversion symmetry, such as KH₂PO₄ (KDP), KD₂PO₄ (KD*P or DKDP), lithium niobate (LiNbO₃), beta-barium borate (BBO), barium titanate (BTO), and (3) EO polymers, including non-linear optic (NLO) chromophore-polymer composite materials.

EO materials containing liquid crystals generally have desirable EO coefficient but exhibit inherently low phase modulation speeds due to the parasitic effect of the crystal metastructure. Conversely, EO materials containing lithium niobate and/or other inorganic crystals generally achieve desirable modulation speeds but their EO effects are inherently limited by optically active point defects invariably formed in the crystals during growth.

NLO chromophore-polymer composite materials can provide both high EO coefficient and high modulation speeds. However, stability of the NLO chromophore and polymer constituents is a key challenge to the development of EO polymer materials for practical use in commercial EO devices. To satisfy the stringent requirements for such devices, NLO chromophores in particular should be both photostable under photolytic and photooxidative processing conditions and resistant to thermal decay under long duration use processing temperatures. Accordingly, a need exists for the development of NLO chromophores with not only high electro-optic activity, but also robust photo- and thermal stability under environmental conditions.

### BRIEF SUMMARY

The present disclosure is directed, in general, to (1) nonlinear optical (NLO) chromophores, including (2) compositions/materials/resistive layers comprising NLO chromophores, and the methods of making the compositions/materials/resistive layers comprising NLO chromophores (e.g., methods of poling and/or drying, and the like), (3) uses of NLO chromophores in electro-optic devices (e.g., EOMs). NLO chromophores disclosed herein not only have large EO effect, but also have fast modulation speed. In addition, NLO chromophores disclosed herein have superior photostability and thermal stability compared to other EO Materials. As a consequence, NLO chromophores herein are particularly suited for use as EO materials in connection with low power and small footprint devices, including devices used in data acquisition systems, analog I/O modules, field transmitters, lab and field instrumentation, servo drive control modules, direct current (DC) power supply, alternating current (AC), and/or electronic load.

In one aspect, NLO chromophores containing spirofluorene-isophorone bridging groups are disclosed. In one example, various embodiments of the present disclosure include nonlinear optical chromophores of the general formula (I):

D-Π-A (I)
wherein D represents an organic electron-donating group; A represents an organic electron-accepting group having an electron affinity greater than the electron affinity of D; and Π represents a Π-bridge between the organic electron-accepting group and the organic electron-donating group; wherein the Π-bridge has the following formula (Π¹):

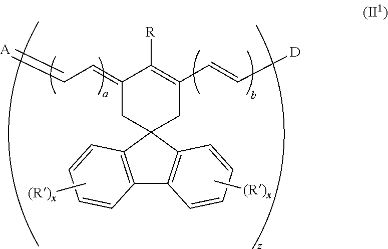

wherein a and b independently each represent an integer of 1 to 3; wherein each x independently represents an integer of 0 to 4; wherein z represents an integer of 1 to 3; wherein each R′ independently represents a substituent selected from the group consisting of hydrogen, halogen, a substituted or unsubstituted aryl, a substituted or unsubstituted alkyl, a substituted or unsubstituted alkylaryl, a substituted or unsubstituted carbocyclic, a substituted or unsubstituted heterocyclic, and a substituted or unsubstituted cyclohexyl; wherein each R independently represents a substituent selected from the group consisting of a hydrogen, a substituted or unsubstituted aryl, a substituted or unsubstituted alkyl, a substituted or unsubstituted sulfhydryl, a substituted or unsubstituted hydroxyl, or a substituted of unsubstituted amino.

Various embodiments of the nonlinear optical chromophore with spirofluorene-isophorone bridge may exhibit high EO coefficient r₃₃. The EO coefficient r₃₃ may be one of the most important EO properties which represents the relationship between the change in applied electric potential versus the change in the refractive index of the material. A higher EO coefficient r₃₃ may indicate that the nonlinear optical chromophore has better EO properties and better potential usage.

In the same or another example, various embodiments of the present disclosure may include nonlinear electro-optic materials that include both the nonlinear optical chromophores described above and one or more matrix material, also referred to as host polymer, in which the one or more nonlinear optical chromophore may be incorporated. The nonlinear optical chromophore may generally be incorporated within the matrix material in virtually any amount, or can be used with no matrix material (i.e., “neat” or 100% chromophore).

In the same or another example, various embodiments of the present disclosure may include compositions that include both the nonlinear electro-optic material described above and solvents. Solvents which are suitable for use in the various embodiments may include regular solvents and/or high boiling point solvents. High boiling point solvents may include solvents having a boiling point greater than or equal to 100° C. (at 1 atm). The glass transition temperature (T_{g}), in general, is the temperature at which an amorphous polymer changes from a hard/glassy state to a soft/rubbery state, or vice versa.

In the same or another example, various embodiments of the present disclosure may include compositions having nonlinear optical chromophores with spirofluorene-isophorone bridge. The compositions having nonlinear optical chromophores with spirofluorene-isophorone bridge may exhibit high stabilities, including both photostability and thermal stability. The thermal stability of a nonlinear optical chromophore may be evaluated based on a decomposition temperature (Td) of the chromophore, and/or a thermal decay of the chromophore. For example, the decomposition temperature (Td) may be the temperature at which the chromophore chemically decomposes. For another example, the thermal decay may be the percentage of chemically decomposed chromophore under a given temperature for a given period of time.

Meanwhile, a high photostability ensures the nonlinear optical chromophore will not degrade under illumination in an air atmosphere. The photostability of a nonlinear optical chromophore may be evaluated based on a photo decay. For example, the photo decay may be the percentage of degraded chromophore after exposing the nonlinear optical chromophore under UV-Vis in a given period of time.

In the same or another example, various embodiments of the present disclosure include resistive layers formed from the compositions described above through one or more procedures. The one or more procedures may include, but not limited to, poling and/or drying.

During the poling and/or drying process, an electro-optic material may be dispersed in a suitable solvent in virtually any amount that provides a homogenous solution and suitable properties for resistive layer formation. The resistive layers may be poled by applying a suitable voltage across the material at a suitable temperature. After poling the resistive layer, while still maintaining the field of applied voltage, the resistive layers may be dried and/or densified by removing the remaining solvent.

In the same or another example, various embodiments of the present disclosure may include electro-optic devices with electro-optical functions that contain one or more resistive layers described above. The electro-optic devices may include electro-optic modulators (EOMs), which are optical devices in which a signal-controlled element exhibiting an electro-optic effect is used to modulate a beam of light. In EOMs, the nonlinear electro-optic material may be spun onto silicon wafers, and standard microfabrication techniques may be used to deposit and pattern metal electrodes and optical waveguides.

EOMs comprising various embodiments of nonlinear optical chromophores with spirofluorene-isophorone bridge may include modulators applied in, for example, slot modulators (e.g., slot modulators for wafer-level poling), photonic integrated circuits (e.g., polymer photonic integrated circuits), datacenter switching, high voltage sensing equipment relevant to electric power industry, electrical-to-optical signal transduction equipment which transmits multiple television signals relevant to cable television (CATV) or satellite television, broad bandwidth acoustic spectrum analyzers, optical gyroscopes, phased array radar (e.g., integrated antenna/electro-optic modulator or w-band optical modulator), photonically detected radar, time stretching and ultrafast analog-to-digital conversion equipment, components for fiber optical and satellite telecommunications, generation equipment and detection equipment of ultrafast electrical fields, electric field sensor (e.g., electro-optic E-field sensor), land mine detection equipment, device related to wavelength division multiplexing, optical switching, devices related to spatial light modulation (e.g., devices related to beam steering), and/or augmented reality (AR)/virtual reality (VR) equipment (e.g., full-spectrum visible electro-optic modulator).

For example, the photonic integrated circuit (PIC) may be a chip that performs optical signal processing. The chip may contain two or more photonic components (e.g., resistive layer with nonlinear electro-optic materials) which form a functioning circuit to utilize photons to detect, generate, transport, and process light. The PICs have demonstrated huge potentials in delivering the performance (e.g., speed, size and efficiency) required for upcoming applications, such as 6G, automotive light detection and ranging (LiDAR), consumer healthcare, artificial intelligence (AI), optical computing, virtual reality (VR), and/or augmented reality (AR).

### BRIEF DESCRIPTION OF SEVERAL VIEWS OF THE DRAWINGS

The foregoing summary, as well as the following detailed description of preferred embodiments of the disclosure, will be better understood when read in conjunction with the appended drawings. The embodiments of the drawings are shown for illustration. It should be understood, however, that the disclosure is not limited to the precise arrangements and instrumentalities shown.

In the drawings:

FIG. 1 is a depiction of a degenerate four wave mixing (DFWM) testing procedure for evaluation of third-order NLO properties of thin film composites.

FIG. 2 illustrates an example poling process of a nonlinear electro-optic material.

FIG. 3A is a partial side sectional diagram of an integrated electro-optic circuit.

FIG. 3B is a partial cross-sectional diagram of the integrated electro-optic circuit of FIG. 3A.

FIG. 4 is an end view of a slot modulator with highly doped silicon slab and rail.

### DETAILED DESCRIPTION

### Terms and Concepts

As used herein, the term “nonlinear optical chromophore” (NLO chromophore) refers to molecules or portions of a molecule that create a nonlinear electro-optic effect when irradiated with light.

As used herein, the term “electron-donating group” refers to an atom and/or a functional group that donates some of its electron density into a conjugated H system via resonance and/or inductive effects.

As used herein, the term “electron-accepting group” refers to an atom and/or a functional group that accepts some of the electron-donating group's electron density in a conjugated Π system via resonance and/or inductive effects.

As used herein, the term “bridging group” refers to a functional group that bridges between the electron-donating group and the electron-accepting group in a conjugated Π system.

As used herein, the term “four-wave mixing” (FWM) refers to an interaction of four spatially or spectrally distinct fields.

As used herein, the term “r₃₃” refers to an electro-optic coefficient, a function of a first-order hyperpolarizability, that represents the relationship between the change in applied electric potential versus the change in the refractive index of the material. The “r₃₃” is expressed in units of pm/V. The “r₃₃” is the principal element of the Pockels EO effect tensor and is a function of first-order hyperpolarizability (β) which denotes the magnitude of refractive index shift (Δη) obtained for an applied low-frequency electric field that represents the relationship between the change in applied electric potential versus the change in the refractive index of the material.

As used herein, the term “c” refers to a dielectric constant, which is also known as permittivity. The dielectric constant is a measure of the extent to which a substance is polarized under an applied (external) electric field. Polarization amounts to net separation of charge across the substance.

As used herein, the term “susceptibility” refers to the degree to which a material can be polarized by an external electric field. There are different orders of susceptibility such as linear susceptibility (χ⁽¹⁾), second-order susceptibility (χ⁽²⁾), third-order susceptibility (χ⁽³⁾) and other higher-order susceptibilities. Second-order susceptibility describes the material's response to two electric fields of different frequencies. The electro-optic effect occurs when an electric field is applied to a material with a non-zero second-order susceptibility. When an electric field is applied to such a material, the polarization of the material changes, resulting in a change in the refractive index. The change in the index of refraction and the magnitude of the externally applied electric field is proportional. Third-order susceptibility (χ⁽³⁾) describes the material's response to three electrical fields of different frequencies. The third-order susceptibility coefficient associated with each electrical field will be different due to the ever-present dispersion (i.e. frequency dependence) of the susceptibilities.

As used herein, the terms “optic nonlinearity,” “nonlinearity,” and “nonlinear” refer to the behavior of light in nonlinear media, that is, media in which the polarization density P responds non-linearly to the electric field (E) of the light. The nonlinearity is typically observed only at very high light intensities (when the electric field of the light is >108 V/m and thus comparable to the atomic electric field of ˜1011 V/m) such as those provided by lasers.

As used herein, the term “nonlinear electro-optic material” refers to materials that include both the nonlinear optical chromophore and one or more matrix material, also referred to as host polymer, in which the one or more nonlinear optical chromophore may be incorporated. EO materials can exhibit a nonlinear EO effect. Suitable matrix materials can include polymers, such as, for example: poly(methylmethacrylate)s (PMMA); polyimides; polyamic acid; polystyrenes; poly(urethane)s (PU); and amorphous polycarbonates (APC). NLO materials are anisotropic in the presence of electromagnetic radiation. When the intensity of the electric field is very high, it creates a very large displacement of the electrons in the material from their equilibrium position. As a result of this, anharmonic behavior comes into the picture of electronic oscillation. So the general linear relationship becomes nonlinear. The polarization (P) of the medium is a nonlinear function of the electric field (E) and it could be expressed as follows:

P=χE

Herein, χ is the electrical susceptibility. χ⁽ⁿ⁾ is the tensor quantity and n is the order of the process. (χ⁽¹⁾=linear polarizability, χ⁽²⁾, χ⁽³⁾ . . . =the first, second . . . hyperpolarizability coefficient, etc.). The nonlinearity is observed only at very high light intensities such as those provided by lasers.

As used herein, the term “compositions” refers to one or more mixed composition(s) that may include both a nonlinear electro-optic material and solvents.

As used herein, the term “resistive layer” refers to one or more layer(s) that may be formed from the compositions defined above through one or more procedures.

As used herein, the term “electro-optic devices” refers to devices with electro-optical function that contain one or more resistive layer(s) described above. For example, the electro-optic devices may include electro-optic modulators (EOMs), which are optical devices in which a signal-controlled element exhibiting an electro-optic effect is used to modulate a beam of light.

As used herein, the term “refractive index” of an optical medium is a dimensionless number that gives the indication of the light bending ability of that medium. The refractive index may determine how much the path of light is bent, or refracted, when entering a material, as described by Snell's law of refraction, n₁ sin θ₁=n₂ sin θ₂, where θ₁ and θ₂ are the angle of incidence and angle of refraction, respectively, of a ray crossing the interface between two media with refractive indices n₁ and n₂. The refractive indices also determine the amount of light that is reflected when reaching the interface, as well as the critical angle for total internal reflection, their intensity (Fresnel's equations) and Brewster's angle. The refractive index may also reflect the factor by which the speed and the wavelength of the radiation are reduced with respect to their vacuum values: the speed of light in a medium is v=c/n, and similarly the wavelength in that medium is λ=λ₀/n, where λ₀ is the wavelength of that light in vacuum. This implies that vacuum has a refractive index of 1 and assumes that the frequency (f=v/λ) of the wave is not affected by the refractive index.

As used herein, the term “electro-optic (EO) effect” is the modification of the optical phase delay (i.e., refractive index) of a medium, caused by an electric field. The strength of an EO effect is correlated with the strength of the material's Pockels effect. The Pockels effect (or linear EO effect) is a directionally dependent linear variation in the refractive index of an optical medium that occurs in response to the application of an electric field. Macroscopically, the Pockels coefficient r relates the change in the index of refraction to an applied electric field as:

$$n ( E ) = {n}_{0} - \frac{1}{2} {rn}_{0}^{3} E$$

where n₀ is the index of refraction under no field and n is the index of refraction under a given electrical field with the voltage equals to E. The applied electrical field shifts the electron cloud to the excited-state molecular orbitals, which alters the refractive index of the EO materials. In optical media, the Pockels effect causes changes in birefringence that vary in proportion to the strength of the applied electric field.

### Nonlinear Optical Chromophore

An electro-optic (EO) effect is a change in the optical properties of a material in responses to an electric field that varies slowly compared with the frequency of light. For example, the electro-optic effect may indicate that a refractive index changes under an electric field. The refractive index change under the electric field may be explained through Pockels effect. Under Pockels effect, the electric field may shift the electron cloud to excited-state molecular orbitals and alter the refractive index of the material, which in turn may cause a phase change to any transiting optical signal.

Materials having the electro-optic effect may include liquid crystals, lithium niobate and/or other inorganic crystals, and/or organic nonlinear optical chromophores. Liquid crystals may have large EO effect but may be slow in modulation speed. Lithium niobate and/or other inorganic crystals may be fast in modulation speed but may have small EO effect. By comparing organic nonlinear optical chromophores with liquid crystals and lithium niobate and/or other inorganic crystals, organic nonlinear optical chromophores may have both large EO effect and fast modulation speed.

As used herein, the term “nonlinear optical chromophore” (NLO Chromophore) refers to molecules or portions of a molecule that create a nonlinear electro-optic effect when irradiated with light. The chromophores are any molecular unit whose interaction with light gives rise to the nonlinear optical effect. The desired effect may occur at resonant or nonresonant wavelengths. The activity of a specific chromophore in a nonlinear electro-optic material is stated as its electro-optic coefficient (r₃₃), which is related to the molecular dipole moment and hyperpolarizability. The various embodiments of NLO chromophores of the present disclosure are useful structures for the production of NLO effects.

Nonlinear optical chromophores in accordance with the various embodiments of the disclosure have the general formula (I):

D-Π-A (I)

wherein D represents an organic electron-donating group; A represents an organic electron-accepting group having an electron affinity greater than the electron affinity of D; and Π represents a Π-bridge between A and D. The terms electron-donating group (donor or “D”), Π-bridge (bridging group or “Π”), and electron-accepting group (acceptor or “A”), and general synthetic methods for forming D-Π-A chromophores are well known in the art.

A donor is an atom or group of atoms that has a low oxidation potential, wherein the atom or group of atoms can donate electrons to an acceptor through a H-bridge. The donor (D) has a lower electron affinity than the acceptor (A), so that, at least in the absence of an external electric field, the chromophore is generally polarized, with relatively less electron density on the donor (D). Typically, a donor group contains at least one heteroatom that has a lone pair of electrons capable of being in conjugation with the p-orbitals of an atom directly attached to the heteroatom such that a resonance structure can be drawn that moves the lone pair of electrons into a bond with the p-orbital of the atom directly attached to the heteroatom to formally increase the multiplicity of the bond between the heteroatom and the atom directly attached to the heteroatom (i.e., a single bond is formally converted to double bond, or a double bond is formally converted to a triple bond) so that the heteroatom gains formal positive charge. The p-orbitals of the atom directly attached to the heteroatom may be vacant or part of a multiple bond to another atom other than the heteroatom. The heteroatom may be a substituent of an atom that has π bonds or may be in a heterocyclic ring. Exemplary donor groups include but are not limited to R₂N— and, RₙX¹—, where R is alkyl, aryl or heteroaryl, X¹ is O, S, P, Se, or Te, and n is 1 or 2. The total number of heteroatoms and carbons in a donor group may be about 30, and the donor group may be substituted further with alkyl, aryl, or heteroaryl.

In some embodiments of the present disclosure, D can represent any organic electron donating group, so long as D is bound to the core at two atomic positions on the core other than the two atomic positions at which A is bound to the core such that at least a portion of D forms a ring fused to the core.

Examples of organic electron donating groups suitable for incorporation into the chromophores of general formula (I) include, but are not limited to, the following structures, wherein the dashed lines represent the two atomic positions at which D forms a ring fused to the core:

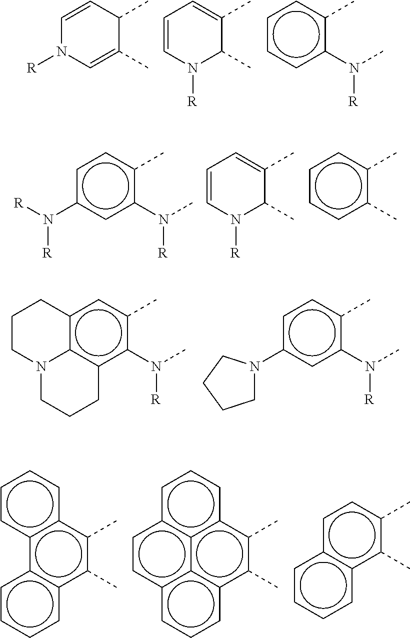

wherein each R independently represents a pendant spacer group.

In various nonlinear optical chromophores in accordance with various embodiments of the present disclosure, suitable electron-donating group include those according to general formula (D^{a}):

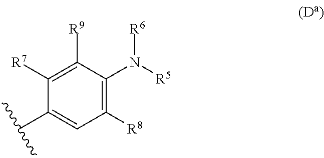

wherein R⁵ represents a moiety selected from the group consisting of H, substituted or unsubstituted linear or branched alkyl, substituted or unsubstituted linear or branched alkenyl, substituted or unsubstituted linear or branched alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10 and wherein R⁵ together with R⁸ or R⁹ may form a 5 or 6 membered ring. R⁸ and R⁹ are H or form a 5 or 6 membered ring with R⁵;
wherein R⁶ represents a moiety selected from the group consisting of a moiety selected from the group consisting of H, halogens, substituted or unsubstituted heteroaryl, substituted or unsubstituted heteroalkyl, substituted or unsubstituted amino, substituted or unsubstituted alkoxycarbonyl, substituted or unsubstituted thioalkyl, substituted or unsubstituted C₁-C₁₀ linear or branched alkyl, substituted or unsubstituted C₂-C₁₀ linear or branched alkenyl, substituted or unsubstituted C₂-C₁₀ linear or branched alkynyl, substituted or unsubstituted aryl, substituted or unsubstituted alkylaryl, substituted or unsubstituted carbocyclic, substituted or unsubstituted heterocyclic, substituted or unsubstituted cyclohexyl, substituted or unsubstituted silyloxy methyl, and CH₃—(CH₂)ₙ—O—(CH₂)ₙ where n is 1-10; and
wherein R⁷ represents a moiety selected from the group consisting of a moiety selected from the group consisting of H and substituted or unsubstituted alkyl.
In one example, one or both of R⁵ and R⁶ is a substituted or unsubstituted phenyl.

In various embodiments, the electron-donating groups having a general formula (Da) may include quinolinyl groups which may be substituted or unsubstituted, including hydro and alkyl substituents, aryl substituents and combinations thereof. Such quinolinyl groups may have one or more diamondoid groups covalently attached thereto. Such quinolinyl groups may include substituted or unsubstituted tetrahydroquinolinyl groups. For example, the substituted or unsubstituted tetrahydroquinolinyl donor group may have general formula (D¹):

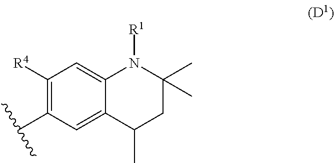

wherein R¹ may include any aryl or alkyl group, and R⁴ may include a hydrogen or a methyl moiety.

In various embodiments, the electron donating group under general formula (D¹) may be:

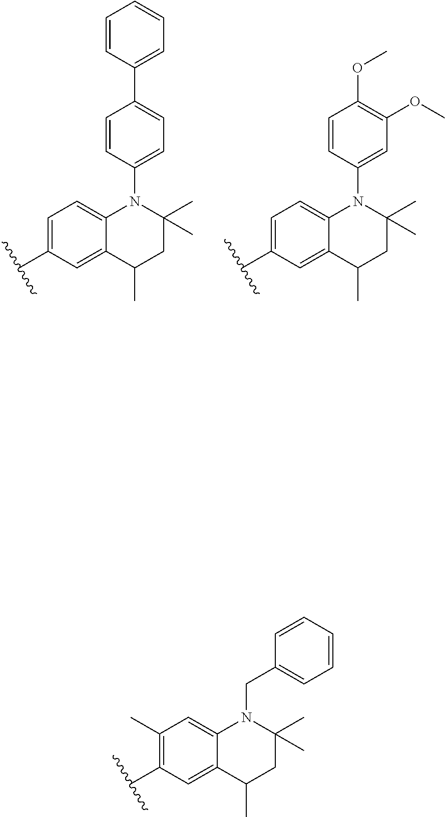

An acceptor is an atom or group of atoms that has a low reduction potential, wherein the atom or group of atoms can accept electrons from a donor through a Π-bridge. The acceptor (A) has a higher electron affinity than the donor (D), so that, at least in the absence of an external electric field, the chromophore is generally polarized in the ground state, with relatively more electron density on the acceptor (D). Typically, an acceptor group contains at least one electronegative heteroatom that is part of a π bond (a double or triple bond) such that a resonance structure can be drawn that moves the electron pair of the π bond to the heteroatom and concomitantly decreases the multiplicity of the π bond (i.e., a double bond is formally converted to single bond or a triple bond is formally converted to a double bond) so that the heteroatom gains formal negative charge. The heteroatom may be part of a heterocyclic ring. Exemplary acceptor groups include but are not limited to —NO₂, —CN, —CHO, COR, CO₂R, —PO(OR)₃, —SOR, —SO₂R, and —SO₃R where R is alkyl, aryl, or heteroaryl. The total number of heteroatoms and carbons in an acceptor group is about 30, and the acceptor group may be substituted further with alkyl, aryl, and/or heteroaryl.

In various nonlinear optical chromophores in accordance with various embodiments of the present disclosure, suitable electron-accepting groups include those according to general formula (A¹):

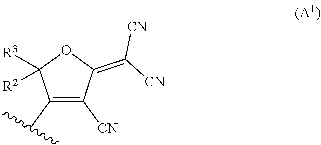

wherein R² and R³ each independently represents a moiety selected from the group consisting of H, substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10. As used herein, [symbol] represents a point of bonding to another portion of a larger molecular structure. In various preferred embodiments, one or both of R² and R³ represent a halogen-substituted moiety. Halogen-substituted may refer to mono-, di-, tri- and higher degrees of substitution. In various preferred embodiments, one of R² and R³ represent a halogen-substituted alkyl moiety and the other represents an aromatic moiety. In various preferred embodiments, one of R² and R³ represent a halogen-substituted aromatic moiety and the other represents an alkyl moiety.

One example of electron-accepting group under general formula (A¹) may be:

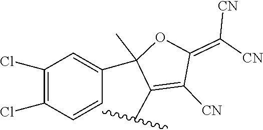

In various embodiments, the above-mentioned R² group in the electron-accepting group may be a trifluoromethyl group. The above-mentioned R³ group in the electron-accepting group may include a substituted or unsubstituted phenyl group, for example, a phenyl group and/or a methylbenzyl group. Therefore, a general formula (A²) of electron-accepting group may be:

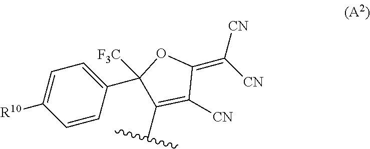

wherein R¹⁰ represents a moiety selected from H, substituted or unsubstituted alkyl, and substituted or unsubstituted aryl.

Examples of electron-accepting group under general formula (A²) may be:

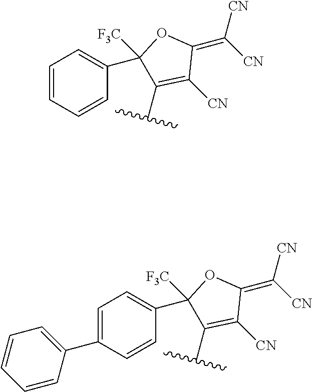

A “Π-bridge” includes an atom or group of atoms through which electrons may be delocalized from an electron donor (defined above) to an electron acceptor (defined above) through the orbitals of atoms in the bridge. Such groups are very well known in the art. Typically, the orbitals will be p-orbitals on double (sp²) or triple (sp) bonded carbon atoms such as those found in alkenes, alkynes, neutral or charged aromatic rings, and neutral or charged heteroaromatic ring systems. Additionally, the orbitals may be p-orbitals on atoms such as boron or nitrogen. Additionally, the orbitals may be p, d or f organometallic orbitals or hybrid organometallic orbitals. The atoms of the bridge that contain the orbitals through which the electrons are delocalized are referred to here as the “critical atoms.” The number of critical atoms in a bridge may be a number from 1 to about 30. The critical atoms may be substituted with an organic or inorganic group. The substituent may be selected with a view to improving the solubility of the chromophore in a polymer matrix, to enhance the stability of the chromophore, or for other purposes.

In various embodiments, bridging groups (H) for nonlinear optical chromophores according to general formula (I) of the present disclosure include a spirofluorene-isophorone bridge of the general formula (II¹):

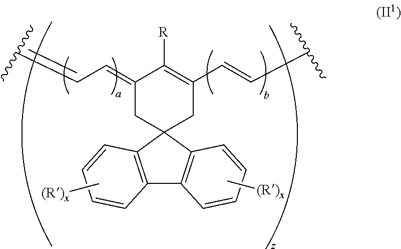

each R′ may represent a substituent selected from the group consisting of a hydrogen a halogen, a substituted or unsubstituted aryl, a substituted or unsubstituted alkyl, a substituted or unsubstituted alkylaryl, a substituted or unsubstituted carbocyclic, a substituted or unsubstituted heterocyclic, and a substituted or unsubstituted cyclohexyl; wherein each R may represent a substituent selected from the group consisting of a hydrogen, a substituted or unsubstituted aryl, a substituted or unsubstituted alkyl, a substituted or unsubstituted sulfhydryl, a substituted or unsubstituted hydroxyl, or a substituted of unsubstituted amino; wherein each a and b independently represents an integer of 0 to 3; each x independently represents an integer of 0 to 4; and z represents an integer of 1 to 3.

Examples of bridging group under general formula (Π¹) may be:

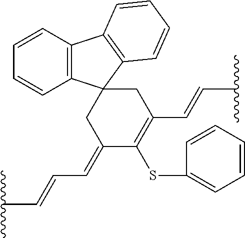

Nonlinear optical chromophores according to the various embodiments of the present disclosure further include a spirofluorene-isophorone bridging group connected to electron donating group and electron accepting group. The spirofluorene-isophorone bridging group may function as a Π-bridge in the chromophore.

Examples of chromophores with a spirofluorene-isophorone bridging group according to the various embodiments of the present disclosure may include the following chromophores:

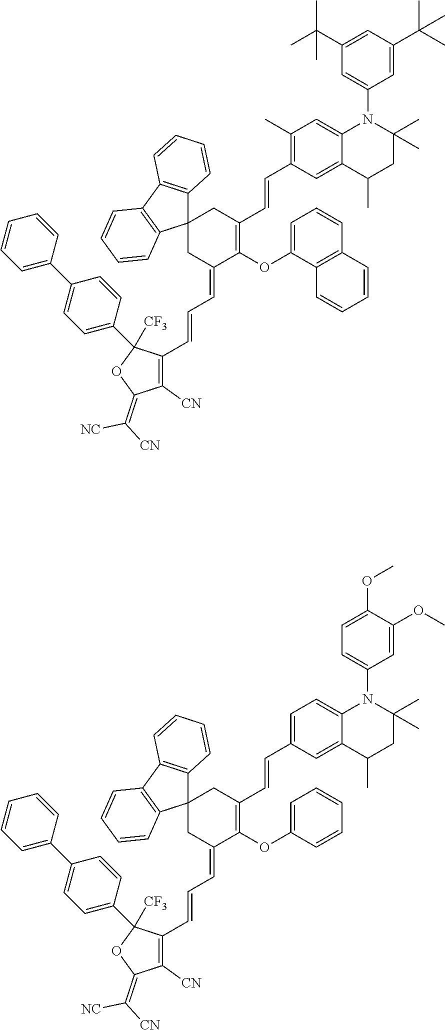 [SMILES: Cc1cc2c(cc1/C=C/C1=C(Oc3cccc4ccccc34)C(=C/C=C/C3=C(C#N)C(=C(C#N)C#N)OC3(c3ccc(-c4ccccc4)cc3)C(F)(F)F)/CC3(C1)c1ccccc1-c1ccccc13)C(C)CC(C)(C)N2c1cc(C(C)(C)C)cc(C(C)(C)C)c1 ; COc1ccc(N2c3ccc(/C=C/C4=C(Oc5ccccc5)C(=C/C=C/C5=C(C#N)C(=C(C#N)C#N)OC5(c5ccc(-c6ccccc6)cc5)C(F)(F)F)/CC5(C4)c4ccccc4-c4ccccc45)cc3C(C)CC2(C)C)cc1OC]

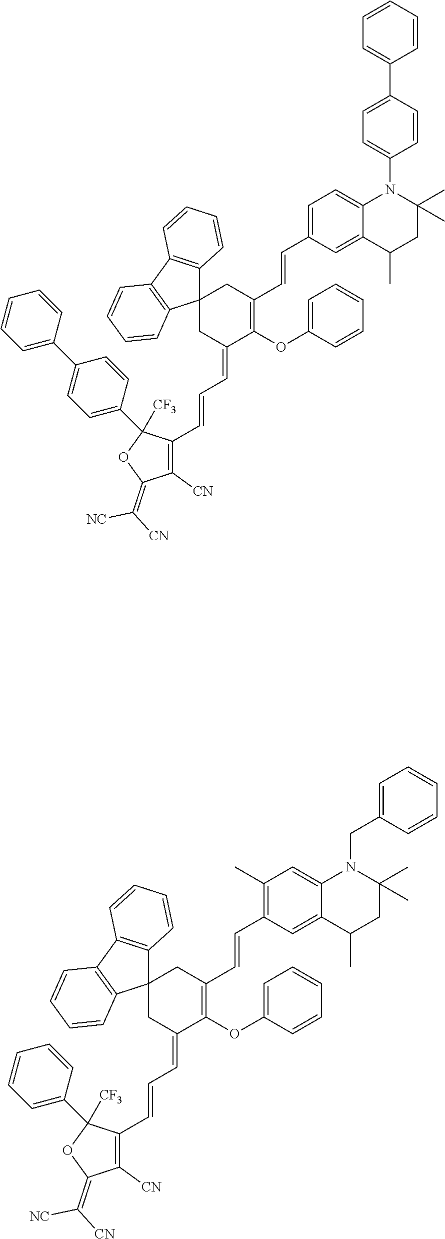 [SMILES: CC1CC(C)(C)N(c2ccc(-c3ccccc3)cc2)c2ccc(/C=C/C3=C(Oc4ccccc4)C(=C/C=C/C4=C(C#N)C(=C(C#N)C#N)OC4(c4ccc(-c5ccccc5)cc4)C(F)(F)F)/CC4(C3)c3ccccc3-c3ccccc34)cc21 ; Cc1cc2c(cc1/C=C/C1=C(Oc3ccccc3)C(=C/C=C/C3=C(C#N)C(=C(C#N)C#N)OC3(c3ccccc3)C(F)(F)F)/CC3(C1)c1ccccc1-c1ccccc13)C(C)CC(C)(C)N2Cc1ccccc1]

The first-order hyperpolarizability (β) is one of the most common and useful NLO properties. An electro-optic coefficient (r₃₃) is a function of β, and a sufficient value of r₃₃ may indict a good electro-optical property in a given NLO. For example, the sufficient value of r₃₃ may be equal to or more than 100 pm/V.

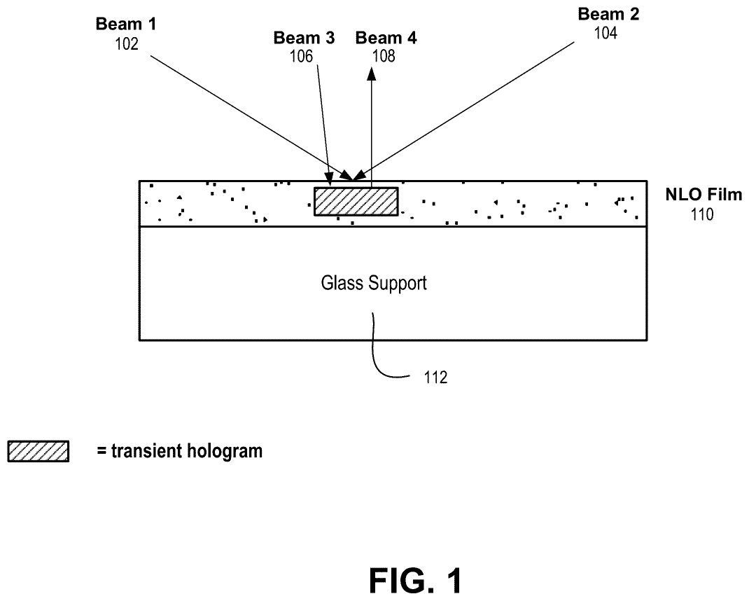

The second-order hyperpolarizability (γ) or third-order susceptibility (χ⁽³⁾), are the normal measures of third-order NLO activity. While there are several methods used to measure these properties, degenerate four-wave mixing (DFWM) is very common. The term four-wave mixing (FWM) is usually reserved for the interaction of four spatially or spectrally distinct fields. In most common FWM processes, some of the frequencies, wave vectors, and polarizations are degenerated. For example, FWM may reduce to most common FWM processes when two or more off the frequencies are degenerate. The most common FWM processes may include, but not limited to, coherent anti-stokes Raman spectroscopy (CARS), coherent stokes Raman spectroscopy (CSRS), stimulated Raman gain spectroscopy (SRS), the inverse Raman effect spectroscopy (TIRES), and/or Raman induced Kerr effect spectroscopy (RIKES). FWM may be used to probe either one-photon resonances or two-photon resonances in a material by measuring the resonant enhancement as one or more of the frequencies are tuned. A method of evaluating third-order NLO properties of thin films, known in the art as degenerate four-wave mixing (DFWM), may be illustrated in FIG. 1. In FIG. 1, Beams 102 and 104 are picosecond, coherent pulses, absorbed by the NLO film 110 deposited on a glass substrate 112. Beam 106 is a weaker, slightly delayed beam at the same wavelength as Beams 102 and 104. Beam 108 is the resulting product of the wave mixing, diffracted off of the transient holographic grating, produced by interferences of beams 102 and 104 in the NLO material of the film 110. Beam 106 can be a “control” beam at a telecom wavelength which produces a “signal” beam at a frequency not absorbed by the NLO material.

The EO property of the poled nonlinear electro-optic material that incorporates nonlinear optical chromophore may be tested as follows. Polarized light, often from a laser, is passed through the poled material that incorporates the poled nonlinear optical chromophore, then through a polarizing filter, and to a light intensity detector. If the intensity of light received at the detector changes as the electric potential applied to the electrodes is varied, the material incorporates a nonlinear optic chromophore and has an electro-optically variable refractive index.

The relationship between the change in applied electric potential versus the change in the refractive index of the material may be represented as its EO coefficient r₃₃. This effect is commonly referred to as an electro-optic, or EO, effect. Devices that include materials that change their refractive index in response to changes in an applied electric potential are called electro-optical (EO) devices. For compositions having nonlinear optical chromophore with spirofluorene-isophorone bridge described herein, the EO coefficient r₃₃ of >45 pm/V, or >75 pm/V, or >110 pm/V, or even larger may be achieved.

### Nonlinear Electro-Optic Material

As used herein, the term “nonlinear electro-optic material” refers to materials that include both the nonlinear optical chromophore and one or more matrix material, also referred to as host polymer, in which the one or more nonlinear optical chromophore may be incorporated. Suitable matrix materials can include polymers, such as, for example: poly(methylmethacrylate)s (PMMA); polyimides; polyamic acid; polystyrenes; poly(urethane)s (PU); and amorphous polycarbonates (APC).

Glass transition temperature (T_{g}) is a temperature at which an amorphous polymer changes from a hard/glassy state to a soft/rubbery state, or vice versa. In various embodiments the matrix material can comprise a poly(methylmethacrylate), for example having a molecular weight of about 120,000 and a glass transition temperature T_{g} of about 100-165° C., or an APC having a T_{g} of about 150-220° C.

The nonlinear optical chromophore can generally be incorporated within the matrix material in virtually any amount, or can be used with no matrix material (i.e., “neat” or 100% chromophore). For example, suitable electro-optic material can comprise a nonlinear optical chromophore in an amount of from about 1% to 90% by weight, based on the entire weight of combined nonlinear optical chromophores and matrix materials. In various embodiments, suitable electro-optic compositions can comprise a nonlinear optical chromophore in an amount of from about 2% to 80% by weight, based on the entire weight of combined nonlinear optical chromophores and matrix materials. In various embodiments, suitable electro-optic compositions can comprise a nonlinear optical chromophore in an amount of from about 3% to 75% by weight, based on the entire weight of combined nonlinear optical chromophores and matrix materials. For example, one or more chromophores can be combined with an amorphous polycarbonate or mixtures of matrix materials at 70 wt % chromophore(s)/30 wt % matrix material(s). In various embodiments, chromophores can be crosslinked with matrix materials or other polymers.

### Compositions

As used herein, the term “compositions” refers to one or more mixed composition(s) that may include both a nonlinear electro-optic material and solvents. Solvents which are suitable for use may include regular boiling point solvents and high boiling point solvents. As used herein, “high boiling point solvents” refers to solvents having a boiling point greater than or equal to 100° C. (at 1 atm). In various embodiments, suitable solvents have a boiling point greater than or equal to 110° C., greater than or equal to 120° C., greater than or equal to 130° C., greater than or equal to 140° C., greater than or equal to 150° C., greater than or equal to 160° C., greater than or equal to 170° C., greater than or equal to 180° C., greater than or equal to 190° C., greater than or equal to 200° C., greater than or equal to 210° C., greater than or equal to 220° C., greater than or equal to 230° C., greater than or equal to 240° C., and greater than or equal to 250° C.

Suitable solvents for use in the various embodiments are capable of forming a homogenous solution for the compositions, and generally can include high boiling point, relatively nonpolar, aprotic solvents. Suitable solvents include, for example, N-methylpyrrolidone, dimethylsulfoxide, carbonates such as ethylene carbonate and propylene carbonate, and glycol ethers such as diethylene glycol dibutyl ether. Solvents considered “polar,” such as DMSO, can be used and considered relatively nonpolar to the extent they can dissolve both polar and nonpolar solutes. In various embodiments, a suitable high boiling point solvent can include diethylene glycol dibutyl ether. In various embodiments, a high boiling point solvent can be used in admixture with a co-solvent that does not have a high boiling point.

Nonlinear optical chromophore with spirofluorene-isophorone bridge, as well as compositions having nonlinear optical chromophore with spirofluorene-isophorone bridge described herein may exhibit high stabilities, which may include both photostability and thermal stability. The thermal stability of a nonlinear optical chromophore may be evaluated based on a decomposition temperature (Td) of the chromophore, and/or a thermal decay of the chromophore. The decomposition temperature (T_{d}) is the temperature at which the chromophore chemically decomposes. For example, the nonlinear optical chromophore with spirofluorene-isophorone bridge described herein may have a decomposition temperature greater than or equal to 200° C., or greater than or equal to 225° C., or greater than or equal to 250° C., or even higher. The thermal decay is the percentage of chemically decomposed chromophore under a given temperature for a given period of time. For example, the nonlinear optical chromophore with spirofluorene-isophorone bridge described herein may have a thermal decay <15%, or <10%, or <5%, or even lower under 180° C. in 90 minutes.

The photostability is one of the most useful properties of the nonlinear optical chromophores. It is important for chromophores to have high photostability because high photostability ensures the chromophores will not degrade under illumination in an air atmosphere. The degradation of chromophores under illumination in the air will happen if double bonds in the nonlinear optical chromophores react with molecular oxygen. The molecular oxygen in the air atmosphere may be either triplet oxygen or singlet oxygen. Triplet oxygen is the electronic ground state of molecular oxygen, which means triplet oxygen is the most stable and common allotrope of oxygen and is not reactive toward the double bonds. Light converts triplet oxygen molecules to singlet oxygen molecules, which are very reactive toward double bonds. In summary, singlet oxygen is the reason why the chromophores will degrade under illumination in the air atmosphere. The reaction of singlet oxygen with the double bonds of chromophores will deactivate the chromophores by making the double bonds no longer optically active. Therefore, if a nonlinear optical chromophore is resistant to the degradation by singlet oxygen, the nonlinear optical chromophore will have high photostability and less resources have to be spent on excluding oxygen from the working device.

The photostability of a chromophore may be evaluated based on the photo decay. Photo decay is the percentage of degraded chromophore after exposing the nonlinear optical chromophore under UV-Vis in a given period of time. For example, the nonlinear optical chromophore with spirofluorene-isophorone bridge described herein may have a photo decay <15%, or <10%, or <5%, or even lower after exposing under light for 20 hours.

Nonlinear optical chromophores of embodiments having a spirofluorene-isophorone bridging group exhibit high photostability. For example, the nonlinear optical chromophore with spirofluorene-isophorone bridging group may have a photo decay <15%, or <10%, or <5%, or even lower after exposing under broadband UV-Vis for 20 hours. The photo decay is measured by calculating the percentage of chromophore degraded via UV-Vis after putting the chromophore under illumination using a 6 mW broadband lamp (e.g., 360 nm to 2600 nm wavelength range) in ambient temperature and pressure for 20 hours. For example, 45% photo decay means 45% of the nonlinear optical chromophores are degraded by the light and/or singlet oxygen while 55% of the chromophores are unchanged under illumination for 20 hours. Therefore, a low photo decay may indicate a high photostability of the chromophores.

Photo decay data of various embodiments in nonlinear optical chromophores having a spirofluorene-isophorone bridging group is summarized as follows:

| Chromophores | Photo Decay |
|---|---|
| Chromophore A | 9.3% |
| Chromophore B | 7.6% |
| Chromophore C | 4.2% |
| Chromophore D | 8.6% |
| Chromophore E | 9.4% |

In order to achieve above photo decay data, all chromophores are tested via UV-Vis under illumination using a 6 mW broadband lamp (e.g., 360 nm to 2600 nm wavelength range) in ambient temperature and pressure for 20 hours.

Nonlinear optical chromophores of embodiments having a spirofluorene-isophorone bridging group exhibit high thermal stability. In one example, the nonlinear optical chromophore with spirofluorene-isophorone bridging group described herein may have a decomposition temperature (Td) greater than or equal to 200° C., or greater than or equal to 225° C., or greater than or equal to 250° C., or even higher. In another example, the nonlinear optical chromophore with spirofluorene-isophorone bridge described herein may have a thermal decay <15%, or <10%, or <5%, or even lower when being exposed to pure nitrogen atmosphere in ambient pressure under 180° C. in 90 minutes. For example, 5% thermal decay means 5% of the chromophores are decomposed or degraded while 95% of the chromophores are unchanged under 180° C. in 90 minutes. Therefore, a low thermal decay and a high Td may indicate a high thermal stability of the chromophores.

Thermal decay data, T_{d}, as well as glass transition temperature (T_{g}) of various embodiments in nonlinear optical chromophores having a spirofluorene-isophorone bridging group is summarized as follows:

| | Thermal decay | | |
|---|---|---|---|
| Chromophores | (180° C.) | T_{d} | T_{g} |
| Chromophore A | 6.7% | 285° C. | 166° C. |
| Chromophore B | 3.6% | 244° C. | 188° C. |
| Chromophore C | 4.8% | 250° C. | 192° C. |
| Chromophore D | 4% | 255° C. | 189° C. |
| Chromophore E | 3% | 256° C. | 192° C. |

In order to achieve above thermal decay data, all chromophores are tested under 180° C. in 90 minutes when being exposed to pure nitrogen atmosphere in ambient pressure.

### Methods of Forming Resistive Lavers

As used herein, the term “resistive layer” refers to one or more layer(s) that may be formed from the compositions defined above through one or more procedures. The one or more procedures may include, but not limited to, spin-coating and/or an atomic layer deposition (ALD) process. Spin-coating may be a procedure to deposit nonlinear electro-optic material onto flat substrates to form resistive layers. For example, a small amount of nonlinear electro-optic material may be applied on the center of the substrate. The substrate may be rotated at speeds up to 10,000 rpm to spread the nonlinear electro-optic material to form resistive layers by centrifugal force. ALD may be an ultrathin film deposition technique controlled by gas phase and sequential self-limiting chemical reactions of the precursors at the material surface.

In addition, the composition may go through drying and/or poling before the one or more procedures to achieve the desired EO effect. An electro-optic material can be dispersed in a suitable solvent in virtually any amount that provides a homogenous solution and suitable properties for resistive layer formation. For example, the solids content of an electro-optic material in a solvent according to various embodiments described herein can be adjusted depending upon desired resistive layer thickness and spin speed of a spin coating apparatus. As known in the art, a less viscous solution generally results in a thinner spin coated resistive layer. In various embodiments, the solids content of an electro-optic material in a solvent can be from about 1% to about 25%. In various embodiments, the solids content of an electro-optic material in a solvent can be from about 2% to about 20%. In various embodiments, the solids content of an electro-optic material in a solvent can be from about 5% to about 15%.

Example methods in accordance with various embodiments of the present disclosure include providing a composition as described herein, forming a resistive layer comprising the composition, drying the resistive layer (i.e., removing solvent), and poling the resistive layer.

A suitable resistive layer can be formed on a substrate using, for example, a spin-coating process or ink jet printing. Suitable substrates can include indium-tin-oxide (ITO) coated surfaces, conductive materials, silicon, semi-conductors and the like. Resistive layers can be formed at various thicknesses from submicron to several microns.

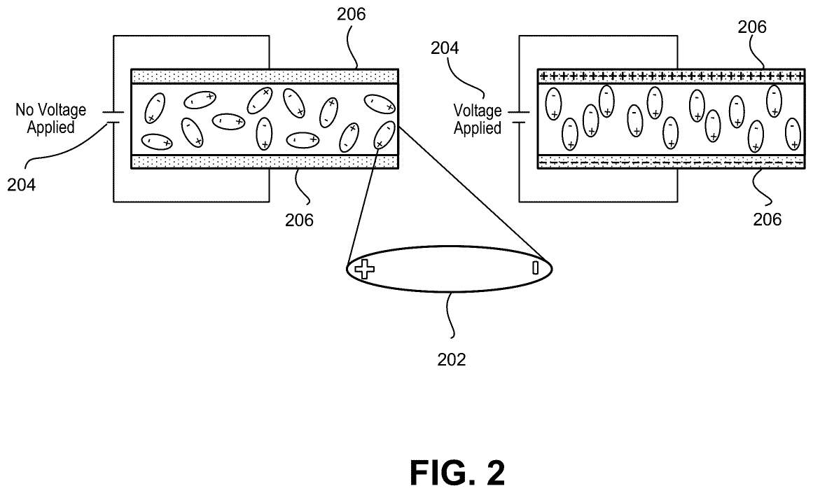

FIG. 2 illustrates an example poling process of a nonlinear electro-optic material. Since the electron density is not evenly distributed inside the nonlinear optical chromophore, the electron density of the electron-accepting group is higher than the electron density of the electron-donating group. Therefore, each nonlinear optical chromophore molecule may comprise a dipole 202, which exhibits positive charge on the electron-donating group side and negative charge on the electron-accepting group side. When no voltage 204 is applied, there is no charge on electrodes 206. The dipoles are randomly directed with no alignment. However, when voltage 204 is applied, electrodes 206 may have charges (e.g., positive charges or negative charges) on them and form an electrical field in between. The dipoles 202 are poled and aligned under the electrical field to make non-centrosymmetric, nonlinear electro-optic materials.

Resistive layers prepared in accordance with various method embodiments disclosed herein can be poled by applying a suitable voltage across the material at a suitable temperature. Electrodes can be formed or positioned on opposing sides of a resistive layer, or above and below a resistive layer in various devices and structures and a suitable voltage applied across the resistive layer in such a manner. Electrodes can be formed from, for example, gold. Suitable voltages can be from about 50 V/μm to about 150 V/μm. Suitable temperatures for poling the resistive layer are generally higher than the nonlinear optical chromophore's glass transition temperature (T_{g}), which is high enough to allow arrangement of the nonlinear optical chromophore within the material. In addition, the solvent is completely removed from the composition prior to poling.

After poling the resistive layer, while still maintaining the field of applied voltage, a resistive layer in accordance with various embodiments described herein can be dried or densified by removing the remaining solvent. Solvent is generally removed until the glass transition temperature of the resistive layer approaches the T_{g} of the chromophore. Drying or removal of the solvent can be undertaken, for example, by slowly and slightly increasing temperature while the poling field is maintained until solvent is removed, then cooling. Drying or removal of the solvent can be undertaken, for example, by cooling while maintaining the applied poling field to a lower temperature such that de-poling does not occur at a substantial rate and then applying vacuum to remove solvent.

Resistive layers in accordance with the various embodiments herein can be incorporated in various devices including electro-optic devices having open-top or coplanar designs, and devices having permeable layers, opening or the like such that solvent can be driven off after poling. Examples of various devices may include, but not limited to, hybrid electro-optic polymer and TiO₂ double-slot waveguide modulators, ultrabroadband electro-optic modulator based on hybrid silicon polymer dual vertical slot waveguide, plate slot polymer waveguide modulator, electro-optic polymer/TiO₂ multilayer slot waveguide modulators, and/or coplanar electrode polymer modulator.

As discussed above, the first-order hyperpolarizability (β) is one of the most common and useful NLO properties. Higher-order hyperpolarizabilities are useful in other applications such as all-optical (light-switching-light) applications. To determine if a nonlinear electro-optic material, such as a compound or polymer, includes a nonlinear optic chromophore with hyperpolarizability and a sufficient electro-optic coefficient (r₃₃), which is a function of β, the material in the form of a resistive layer is placed in an electric field to align the dipoles. This may be performed by sandwiching a resistive layer of the nonlinear electro-optic material between electrodes, such as indium tin oxide (ITO) substrates, gold films, or silver films, for example. To generate a poling electric field, an electric potential is then applied to the electrodes while the nonlinear electro-optic material is heated to its glass transition (T_{g}) temperature. After a suitable period of time, the temperature is gradually lowered while maintaining the poling electric field. Alternatively, the nonlinear electro-optic material can be poled by corona poling method, where an electrically charged needle at a suitable distance from the resistive layer provides the poling electric field. In either instance, the dipoles in the nonlinear electro-optic material tend to align with the field. Various embodiments according to the present disclosure may include electro-optic materials having a material glass transition temperature greater than or equal to 150° C., or greater than or equal to 175° C., or even higher.

### Electro-Optic Devices

As used herein, the term “electro-optic devices” refers to devices with electro-optical function that contain one or more resistive layer(s) described above. For example, the electro-optic devices may include electro-optic modulators (EOMs), which are optical devices in which a signal-controlled element exhibiting an electro-optic effect is used to modulate a beam of light. The modulation may be imposed on the phase/frequency, amplitude, and/or polarization of the beam. One EOM may conduct one or more (e.g., one or all) modulations among the phase/frequency, amplitude, or polarization modulations.

Phase modulation (PM) is a modulation pattern that encodes information as variations in the instantaneous phase of a carrier wave. The phase of a carrier signal is modulated to follow the changing voltage level (amplitude) of the modulation signal. The peak amplitude and frequency of the carrier signal remain constant, but as the amplitude of the information signal changes, the phase of the carrier changes correspondingly.

Amplitude modulation is a process by which the wave signal is transmitted by modulating the amplitude of the signal. Mach-Zehnder (MZ) interferometer as an example. The MZ interferometer may often be used in integrated optics where the requirements of phase stability are more easily achieved. The beam splitter may divide the laser light into two paths, one of which has a phase modulator. The beams may then be recombined. Changing the electric field on the phase modulating path may then determine whether the two beams interfere constructively or destructively at the output, and thereby control the amplitude or intensity of the exiting light. In one example of the MZ interferometer, the modulator may have two arms of electro-optic material. One arm may have electrodes, where a changing voltage can be applied. The other arm may have no voltage applied.

Polarization modulation in EO materials may be used as a technique for time-resolved measurement of unknown electric fields. Depending on the type and orientation of the EO material, and on the direction of the applied electric field, the phase delay may depend on the polarization direction. For example, EOM used in antenna may conduct the polarization modulation.

In EOMs, the nonlinear electro-optic materials are spun onto silicon wafers and standard microfabrication techniques are used to deposit and pattern metal electrodes and optical waveguides. For example, one well-known EOM device is the above-mentioned Mach-Zehnder interferometer. The light output is changed by changing the relative phase between the two arms. One common trick to double the effect for the same available drive voltage is to drive the two arms in opposite directions (push-pull mode). Nonlinear electro-optic materials have an interesting advantage over most other electro-optic materials which are crystalline. The direction of nonlinear electro-optic materials' electro-optic activity is entirely determined by the direction of the applied poling field. By poling the two arms of the MZ in opposite directions, the resulting device automatically has push-pull operation with a single applied signal.

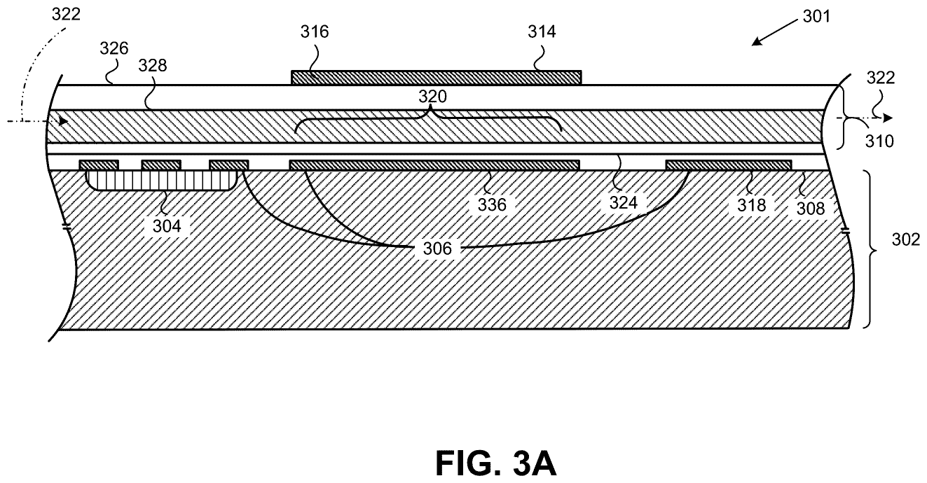

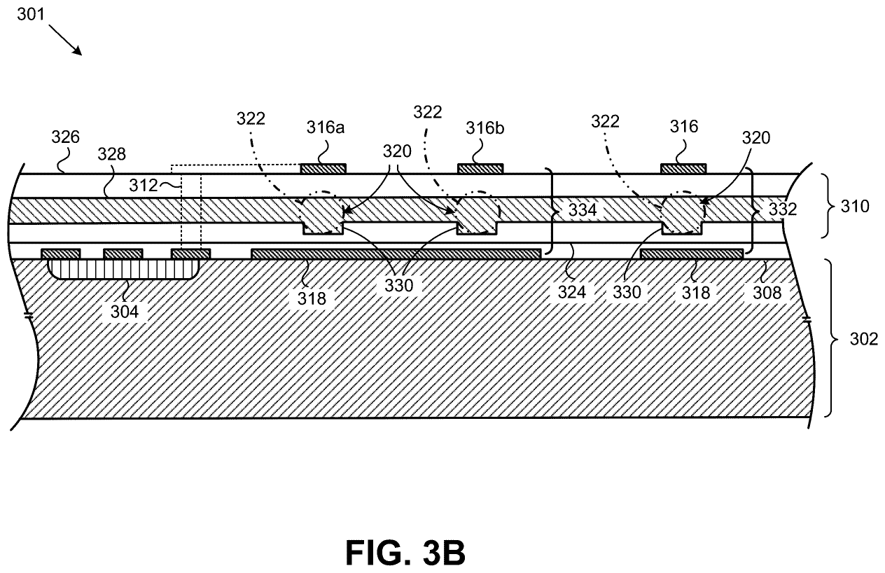

Each EOM may include one or more integrated polymer electro-optic semiconductor circuits. FIGS. 3A and 3B are respective side sectional and cross-sectional views of an integrated polymer electro-optic semiconductor circuit 301, according to an embodiment. A semiconductor substrate 302 includes at least one doping layer 304 patterned across the semiconductor substrate to form portions of semiconductor devices. At least one resistive layer 306 is patterned over the semiconductor substrate. A planarization layer 308 is disposed at least partly coplanar with the at least one conductor layer 306. A polymer optical stack 310 is disposed over the planarization layer 308.

At least one via 312 may at least partially extend through the polymer optical stack 310. The at least one via may be operatively coupled to a corresponding location on the at least one patterned conductor layer 306. A top conductor layer 314 is disposed over the polymer optical stack and in electrical continuity with the at least one via 312.

As an alternative to a via 312, other conductors may be substituted to electrically couple the top conductor layer to at least one location on the at least one patterned conductor layer 306. For example, the at least one conductor may be formed entirely or in combination from a via, a wire bond, a conductive bump, and/or an anisotropic conductive region.

The top conductor layer 314 may be formed to include a metal layer or a conductive polymer, for example. The top conductor may be plated to increase its thickness. The top conductor layer may include at least one high speed electrode 316 formed as a pattern in the top conductor layer 314, the high speed electrode 316 being operatively coupled to receive a signal from the at least one via 312 or other conductive structure from the corresponding location on the at least one patterned conductor layer 306. Thus, the at least one via 312 or other conductive structure is configured to transmit an electrical signal from semiconductor electrical circuitry formed on the semiconductor substrate 302 to the at least one high speed electrode 316 through or around the polymer optical stack 310.

According to embodiments, the at least one patterned conductor layer 306 is configured to form a ground electrode 318 parallel to the at least one high speed electrode 316. An active region 320 of the polymer optical stack 310 is positioned to receive a modulation signal from the high speed electrode 316 and the ground electrode 318. The active region 320 includes a poled region that contains at least one hyperpolarizable organic chromophore.

The polymer optical stack 310 is configured to support the active region 320 as well as receive and guide light 322 to and from the active region. The polymer optical stack 310 may include at least one bottom cladding layer 324 and at least one top cladding layer 326 disposed respectively below and above an electro-optic layer 328. The bottom 324 and top 326 cladding layers, optionally in cooperation with the planarization layer 308, are configured to guide inserted light 322 along the plane of the electro-optic layer 328. Light guiding structures 330 are formed in the polymer optical stack 310 to guide the light 322 along one or more light propagation paths through the electro-optic layer 328 and/or non-active core structures (not shown). In the embodiment of FIGS. 3A and 3B, the guidance structures 330 are formed as trench waveguides that include etched paths in the at least one bottom cladding layer 324.

The integrated polymer electro-optic semiconductor circuit 301 includes a semiconductor electrical circuit formed from a complex of the doping layer pattern 304 and the at least one patterned conductor layer 306. According to an embodiment, the semiconductor electrical circuit is configured, when in operation, to drive the electrodes 316, 318 with a series of modulated electrical pulses. A resultant modulated electrical field is thus imposed across the active region 320 and results in modulated hyperpolarization of the poled organic chromophores embedded therein. A complex of electrodes 316, 318, active region 320 and light guidance structures 330. The modulated hyperpolarization may thus modulate the velocity light passed through the poled active region 320 of the polymer optical stack 310. Repeatedly modulating the velocity of the transmitted light creates a phase-modulated light signal emerging from the active region. Such an active region 320 may be combined with light splitters, combiners (not shown), and other active regions to create light amplitude modulators. Light amplitude modulators herein include MZ optical modulators. Other light amplitude modulators may include ring resonator modulator, which includes one or more ring resonators which is a set of waveguides in which at least one is a closed loop coupled to some light input and output. Other light amplitude modulators may include in-phase and quadrature (I/Q) modulator, which modulates based on the summation of two I/Q signals that are in quadrature.

A combination of at least one electro-optic active region 320, at least two electrodes 316, 318, and corresponding light guiding structures 324, 326, 330 may be considered an electro-optic device 332, 334. A two-channel electro-optic device 334 may be formed from one ground electrode 318 and corresponding pairs of active regions 320 and high speed electrodes 316a, 316b. The two channels of a two channel electro-optic device 334 may operate in cooperation, such as in a push-pull manner to form an MZ optical modulator.

Additional devices may be formed using electrodes or resistors 336 that are not configured for high speed operation. The operation of one such illustrated device is described below in conjunction with the description of an optical phase bias device.

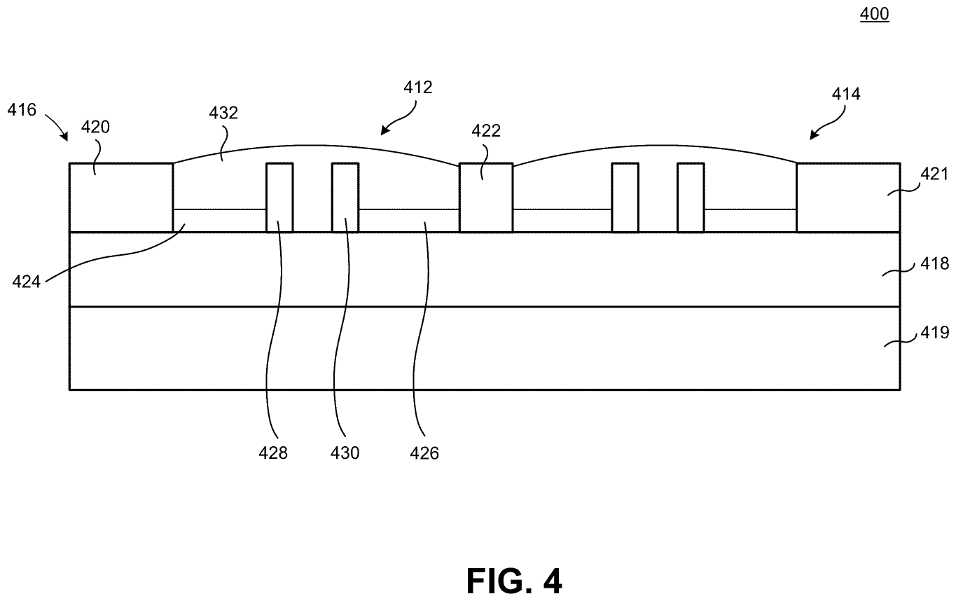

FIG. 4 illustrates an end view of a slot modulator with highly doped silicon slab and rail. Referring specifically to FIG. 4, an end view of a slot modulator 400 is illustrated which in this example is a Mach-Zehnder modulator including two slot waveguides 412 and 414 in parallel and driven in push-pull with a single coplanar transmission line 416. It should be understood that a single slot waveguide can be used to form a slot modulator in accordance with the present invention. In this example, a typical SiO₂ box 418 is formed on a silicon substrate 419. Transmission line 416 is formed of spaced apart aluminum conductors positioned on SiO₂ box 418 with G conductors 420 and 421 on each edge and an S conductor 422 extending midway therebetween. Slot waveguide 412 includes a slab 424 extending inwardly from G conductor 420 and a slab 426 extending inwardly from S conductor 422. A vertically extending rail 428 is attached to the inner end of slab 424 and a vertically extending rail 430, spaced from rail 428, is attached to the inner end of slab 426. Rails 428 and 430 primarily form slot waveguide 412. The area between G conductor 420 and S conductor 422, including the slot formed between rails 428 and 430, is filled with EO polymer cladding material 432. Slot waveguide 414 is a mirror image of slot waveguide 412 with slabs and rails positioned and connected as described in conjunction with slot waveguide 412. In the following disclosure, only slot waveguide 412 is discussed in detail with the understanding that all of the details apply similarly to slot waveguide 414.

To aid in understanding the size of the structure being discussed, the thickness of transmission line 416 is 1 μm, slabs 424 and 426 are each 70 nm tall and 0.5 to 1 μm wide. Rails 428 and 430 are each 220 nm tall (lower surface to upper end) and 240 nm wide with a 200 nm spacing between the centers. The total length of slot waveguide 412 from G conductor 420 to S conductor 422 is 10 um long.

In the prior art, slab 424 and rail 428 are integrally formed and also integrally formed with G conductor 420. Similarly, slab 426 and rail 430 are integrally formed and also integrally formed with S conductor 422. In a similar fashion, the slabs and rails of slot waveguide 414 are integrally formed with G conductor 421 and S conductor 422. In slot modulator 400 slabs 424 and 426 and rails 428 and 430 are formed of silicon that is highly doped (N⁺⁺⁺) to reduce resistivity and to achieve a high bandwidth.

EOMs comprising various embodiments of nonlinear optical chromophores according to the present disclosure may include modulators applied in, for example, slot modulators (e.g., slot modulators for wafer-level poling), photonic integrated circuits (e.g., polymer photonic integrated circuits), datacenter switching, high voltage sensing equipment relevant to electric power industry, electrical-to-optical signal transduction equipment which transmits multiple television signals relevant to cable television (CATV) or satellite television, broad bandwidth acoustic spectrum analyzers, optical gyroscopes, phased array radar (e.g., integrated antenna/electro-optic modulator or w-band optical modulator), photonically detected radar, time stretching and ultrafast analog-to-digital conversion equipment, components for fiber optical and satellite telecommunications, generation equipment and detection equipment of ultrafast electrical fields, electric field sensor (e.g., electro-optic E-field sensor), land mine detection equipment, device related to wavelength division multiplexing, optical switching, devices related to spatial light modulation (e.g., devices related to beam steering), and/or augmented reality (AR)/virtual reality (VR) equipment (e.g., full-spectrum visible electro-optic modulator).

For example, the photonic integrated circuit (PIC) is a chip that performs optical signal processing. The chip may contain two or more photonic components (e.g., resistive layer with nonlinear electro-optic materials) which form a functioning circuit to utilize photons to detect, generate, transport, and process light. The PICs have demonstrated huge potentials in delivering the performance (e.g., speed, size and efficiency) required for upcoming applications, such as 6G, automotive light detection and ranging (LiDAR), consumer healthcare, artificial intelligence (AI), optical computing, virtual reality (VR), and/or augmented reality (AR).

What is claimed is:

1. A nonlinear optical chromophore of the general formula (I):
D-Π-A (I)
wherein D represents an organic electron-donating group; A represents an organic electron-accepting group having an electron affinity greater than the electron affinity of D; and Π represents a Π-bridge between the organic electron-accepting group and the organic electron-donating group; wherein the Π-bridge has the following formula (I): 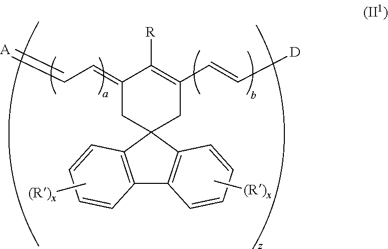
wherein a and b independently each represent an integer of 1 to 3; wherein each x independently represents an integer of 0 to 4; wherein z represents an integer of 1 to 3;
wherein each R′ independently represents a substituent selected from the group consisting of hydrogen, halogen, a substituted or unsubstituted aryl, a substituted or unsubstituted alkyl, a substituted or unsubstituted alkylaryl, a substituted or unsubstituted carbocyclic, a substituted or unsubstituted heterocyclic, and a substituted or unsubstituted cyclohexyl;
wherein each R independently represents a substituent selected from the group consisting of a hydrogen, a substituted or unsubstituted aryl, a substituted or unsubstituted alkyl, a substituted or unsubstituted sulfhydryl, a substituted or unsubstituted hydroxyl, or a substituted of unsubstituted amino.

2. The nonlinear optical chromophore according to claim 1, wherein a, b, and z each represent 1.

3. The nonlinear optical chromophore according to claim 1, wherein x represents 0, and R represents an aryl sulfhydryl substituent.

4. The nonlinear optical chromophore according to claim 2, wherein each R′ independently represents a hydrogen, and R represents an aryl sulfhydryl substituent.

5. The nonlinear optical chromophore according to claim 1, wherein D represents an electron-donating group having general formula (D^{a}): 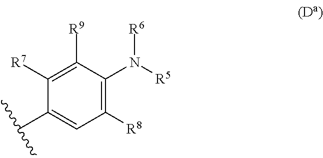
wherein R⁵ represents a moiety selected from the group consisting of H, substituted or unsubstituted linear or branched alkyl, substituted or unsubstituted linear or branched alkenyl, substituted or unsubstituted linear or branched alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10 and wherein R⁵ together with R⁸ or R⁹ may form a 5 or 6 membered ring; R⁸ and R⁹ are H or form a 5 or 6 membered ring with R⁵;
wherein R⁶ represents a moiety selected from the group consisting of a moiety selected from the group consisting of H, halogens, substituted or unsubstituted heteroaryl, substituted or unsubstituted heteroalkyl, substituted or unsubstituted amino, substituted or unsubstituted alkoxycarbonyl, substituted or unsubstituted thioalkyl, substituted or unsubstituted C₁-C₁₀ linear or branched alkyl, substituted or unsubstituted C₂-C₁₀ linear or branched alkenyl, substituted or unsubstituted C₂-C₁₀ linear or branched alkynyl, substituted or unsubstituted aryl, substituted or unsubstituted alkylaryl, substituted or unsubstituted carbocyclic, substituted or unsubstituted heterocyclic, substituted or unsubstituted cyclohexyl, substituted or unsubstituted silyloxy methyl, and CH₃—(CH₂)ₙ—O—(CH₂)ₙ where n is 1-10; and
wherein R⁷ represents a moiety selected from the group consisting of a moiety selected from the group consisting of H and substituted or unsubstituted alkyl.

6. The nonlinear optical chromophore according to claim 5, wherein one or both of R⁵ and R⁶ is a substituted or unsubstituted phenyl.

7. The nonlinear optical chromophore according to claim 1, wherein D represents a quinolinyl electron-donating group having the following formula (D¹): 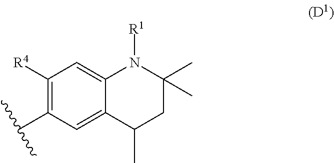
wherein R¹ represents a substituent selected from the group consisting of a substituted or unsubstituted aryl, and a substituted or unsubstituted alkyl group, and R⁴ represents a hydrogen or a methyl moiety.

8. The nonlinear optical chromophore according to claim 1, wherein A represents an electron-accepting group having the following formula (A¹): 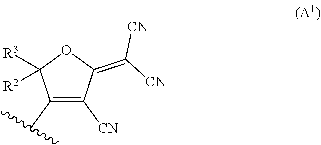
wherein R² and R³ each independently represents a moiety selected from the group consisting of a hydrogen, substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10.

9. The nonlinear optical chromophore according to claim 1, wherein A represents an electron-accepting group having the following formula (A²): 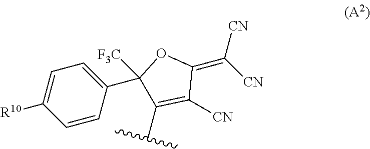
wherein R¹⁰ represents a substituted or unsubstituted phenyl group.

10. A resistive film comprising the nonlinear optical chromophore according to claim 1 dispersed and poled within a matrix material.

11. An electro-optic device comprising one or more resistive film, wherein the one or more resistive film each comprising a nonlinear optical chromophore dispersed and poled within a host polymer matrix, wherein the nonlinear optical chromophore of the general formula (I):
D-Π-A (I)
wherein D represents an organic electron-donating group; A represents an organic electron-accepting group having an electron affinity greater than the electron affinity of D; and Π represents a Π-bridge between the organic electron-accepting group and the organic electron-donating group; wherein the Π-bridge has the following formula (Π¹): 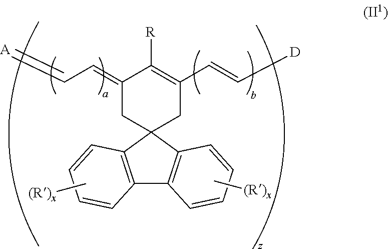
wherein a and b independently each represent an integer of 1 to 3; wherein each x independently represents an integer of 0 to 4; wherein z represents an integer of 1 to 3;
wherein each R′ independently represents a substituent selected from the group consisting of hydrogen, halogen, a substituted or unsubstituted aryl, a substituted or unsubstituted alkyl, a substituted or unsubstituted alkylaryl, a substituted or unsubstituted carbocyclic, a substituted or unsubstituted heterocyclic, and a substituted or unsubstituted cyclohexyl;
wherein each R independently represents a substituent selected from the group consisting of a hydrogen, a substituted or unsubstituted aryl, a substituted or unsubstituted alkyl, a substituted or unsubstituted sulfhydryl, a substituted or unsubstituted hydroxyl, or a substituted of unsubstituted amino.

12. The electro-optic device according to claim 11, wherein a, b, and z each represent 1.

13. The electro-optic device according to claim 11, wherein x represents 0, and R represents an aryl sulfhydryl substituent.

14. The electro-optic device according to claim 12, wherein each R′ independently represents a hydrogen, and R represents an aryl sulfhydryl substituent.

15. The electro-optic device according to claim 11, wherein D represents an electron-donating group having general formula (D^{a}): 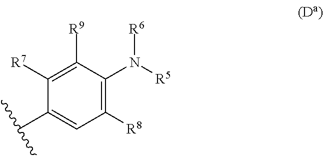
wherein R⁵ represents a moiety selected from the group consisting of H, substituted or unsubstituted linear or branched alkyl, substituted or unsubstituted linear or branched alkenyl, substituted or unsubstituted linear or branched alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10 and wherein R⁵ together with R⁸ or R⁹ may form a 5 or 6 membered ring. R⁸ and R⁹ are H or form a 5 or 6 membered ring with R⁵;
wherein R⁶ represents a moiety selected from the group consisting of a moiety selected from the group consisting of H, halogens, substituted or unsubstituted heteroaryl, substituted or unsubstituted heteroalkyl, substituted or unsubstituted amino, substituted or unsubstituted alkoxycarbonyl, substituted or unsubstituted thioalkyl, substituted or unsubstituted C₁-C₁₀ linear or branched alkyl, substituted or unsubstituted C₂-C₁₀ linear or branched alkenyl, substituted or unsubstituted C₂-C₁₀ linear or branched alkynyl, substituted or unsubstituted aryl, substituted or unsubstituted alkylaryl, substituted or unsubstituted carbocyclic, substituted or unsubstituted heterocyclic, substituted or unsubstituted cyclohexyl, substituted or unsubstituted silyloxy methyl, and CH₃—(CH₂)ₙ—O—(CH₂)ₙ where n is 1-10; and
wherein R⁷ represents a moiety selected from the group consisting of a moiety selected from the group consisting of Π and substituted or unsubstituted alkyl.

16. The electro-optic device according to claim 15, wherein one or both of R⁵ and R⁶ is a substituted or unsubstituted phenyl.

17. The electro-optic device according to claim 11, wherein D represents a quinolinyl electron-donating group having the following formula (D¹): 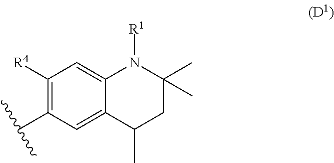
wherein R¹ represents a substituent selected from the group consisting of a substituted or unsubstituted aryl, and a substituted or unsubstituted alkyl group, and R⁴ represents a hydrogen or a methyl moiety.

18. The electro-optic device according to claim 11, wherein A represents an electron-accepting group having the following formula (A¹): 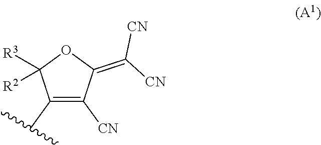
wherein R² and R³ each independently represents a moiety selected from the group consisting of a hydrogen, substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10.

19. The electro-optic device according to claim 11, wherein A represents an electron-accepting group having the following formula (A²): 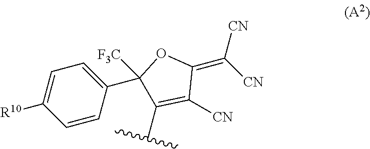
wherein R¹⁰ represents a substituted or unsubstituted phenyl group.
